(19) United States
(12) Patent Application Publication
(10) Pub. No.: US 2008/0033218 A1
(43) Pub. Date: Feb. 7, 2008

(54) Alcohol and olefin production from syngas

(76) Inventors: James R. Lattner, LaPorte, TX (US); Matthew James Vincent, Baytown, TX (US); Kun Wang, Bridgewater, NJ (US); Michel Molinier, Houston, TX (US); Michael J. Veraa, Houston, TX (US); Anthony F. Volpe, Santa Clara, CA (US); Hailian Li, Fremont, CA (US); Jeffrey C. Yoder, San Jose, CA (US); Mark Muraoka, Mountain View, CA (US)

Correspondence Address: EXXONMOBIL CHEMICAL COMPANY, 5200 BAYWAY DRIVE, P.O. BOX 2149, BAYTOWN, TX, 77522-2149, US

(21) Appl. No.: 11/820,754

(22) Filed: Jun. 20, 2007

Related U.S. Application Data
(60) Provisional application No. 60/835,401, filed on Aug. 3, 2006.

(51) Int. Cl. C07C 29/00; B01J 21/04; B01J 23/02

(52) U.S. Cl. 568/897; 502/340; 502/342; 502/344; 502/346; 568/895

(57) ABSTRACT

This invention is directed to a process for making alcohol from syngas, and a process for making olefin, as well as polyolefin, from the alcohol. The syngas is converted to a mixed alcohol stream using a catalyst comprising at least one oxide component. Upon contacting the catalyst with a desired syngas composition, a preferred mixed alcohol product is formed. Preferably, the syngas composition has a stoichiometric molar ratio of less than 2.

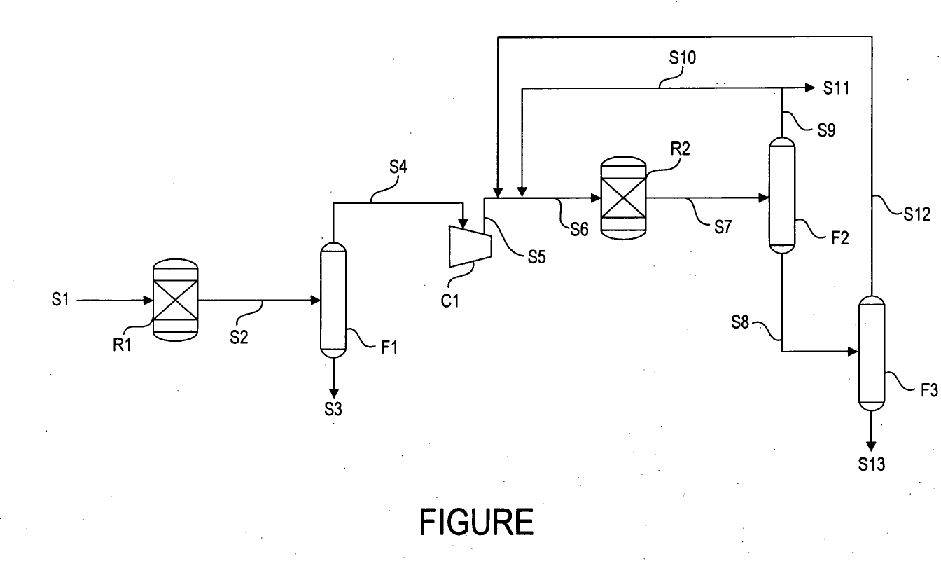

### CROSS REFERENCE TO RELATED APPLICATIONS

This application claims benefit of and priority from U.S. Ser. No. 60/835,401, filed Aug. 3, 2006. The above application is fully incorporated herein by reference.

### FIELD OF THE INVENTION

This invention is directed to a process and catalyst for producing alcohol from syngas. The invention is also directed to making olefin, as well as polyolefin, from the alcohol.

### BACKGROUND OF THE INVENTION

One method of producing olefins such as ethylene and propylene from petroleum-based feedstocks is to use methanol as a feed component. Typically, the synthesis of methanol occurs via the following reaction scheme:

CO+2H₂ [symbol] CH₃OH

CO₂+3H₂ [symbol] CH₃OH+H₂O

The CO and CO₂ are typically obtained from synthesis gas, which is typically derived from the reforming of natural gas.

U.S. Pat. No. 4,499,327 (Kaiser), discloses making olefins from methanol using a variety of SAPO molecular sieve catalysts. The advantage of using SAPO based catalysts, particularly SAPO-34 based catalysts, is that such catalysts produce a substantially large amount of ethylene and propylene relative to other oxygenated hydrocarbons, e.g., alcohols, ethers, etc.

U.S. Pat. No. 6,518,475 (Xu) discloses a process of increasing ethylene selectivity in the conversion of a methanol-based feed. The method includes contacting a silicoaluminophosphate molecular sieve catalyst with a methanol composition that contains from about 1% to about 15% by weight acetone, and separating the ethylene and propylene from the olefin product. The use of the particular feed composition increases the amount of ethylene produced relative to that when pure methanol is used as the feed.

U.S. Patent Publication No. 2004/0116757 (Van Egmond) discloses a methanol-based composition, a method of making the composition, and a method of using the composition as a feedstock. The methanol composition contains methanol as a primary compound, but also includes one or more other alcohols such as ethanol and/or one or more aldehyde compounds. The methanol composition is said to serve as a particularly desirable feed stream for use in the manufacture of olefins such as ethylene and propylene. Such feed streams result in increased production of ethylene or in the increased production of both ethylene and propylene.

U.S. Patent Publication No. 2005/0107482 (Van Egmond) discloses a process for producing light olefins from methanol and ethanol in a mixed alcohol stream. In one embodiment, the invention includes directing a first syngas stream to a methanol synthesis zone to form methanol and directing a second syngas stream and methanol to a homologation zone to form ethanol. The methanol and ethanol are sent to an oxygenate to olefin reaction system for conversion to ethylene and propylene.

Courty, et al., “Production of Methanol-Higher Alcohol Mixtures from Natural Gas Via Syngas Chemistry,” Revue de L'Insitut Francais du.Pétrole, 45 (4) (1990), 561-578, discuss methods of making C₁-C₆ alcohols from syngas. The use of copper-cobalt, copper nickel and rubidium promoted methanol synthesis catalysts is presented. The use of copper rubidium catalyst at low H₂/CO stoichiometry was reported favorable, although alcohol purity was reported as the lowest. The use of copper cobalt catalyst was reported to give the best alcohol purity.

Stiles, et al., “Catalytic Conversion of Synthesis Gas to Methanol and Other Oxygenated Products,” Ind. Eng. Chem. Res., 30 (1991), 811-821, report on test results obtained in converting syngas to oxygenate products using various catalysts, and varying test conditions such as temperature, pressure, gas composition, space velocity and production rate. It was reported that substantial percentages of acetaldehyde, propionaldehyde, isobutyraldehyde and methyl ethyl ketone could be produced, which was viewed as being a potential use for the manufacture of acetic and propionic acids and methyl tert-butyl ether. An alcohol synthesis mechanism was also proposed.

Mixed alcohol streams, especially mixed methanol and ethanol streams, are desirable for use as feed streams in the production of olefins. The synthesis of ethanol and higher alcohol streams typically proceed via similar reaction chemistry to that of methanol, which can be represented by the following reaction scheme:

nCO+2nH₂ [symbol] CₙH₂ₙ₊₁OH+(n−1)H₂O

nCO₂+3nH₂ [symbol] CₓH₂ₙ₊₁OH+(2n−1)H₂O

Streams of mixed alcohol components, such as varying the amount of alcohols such as methanol and ethanol, enable shifting the concentration of ethylene and propylene in the olefin product. Ethanol is particularly desirable at certain levels in order to increase the concentration of ethylene produced in the olefin product. It would, therefore, be desirable to find more efficient processes and catalysts for making mixed alcohol streams that could be used in the production of olefins.

### SUMMARY OF THE INVENTION

This invention provides a process and catalyst for producing mixed alcohol streams, such as a mixed methanol and ethanol stream, and using that stream as a feedstock for the manufacture of olefins, and ultimately polyolefins. In particular, the invention produces an alcohol stream that includes ethanol in a quantity that increases the production of ethylene concentration in the olefin product.

According to one aspect of the invention, there is provided a process for producing alcohol from syngas. The process includes sending a syngas stream to an alcohol synthesis reactor, wherein the syngas stream entering the reactor has a stoichiometric molar ratio of less than 2. The syngas is contacted in the alcohol synthesis reactor with a catalyst comprising copper and an oxide of at least one element selected from the group consisting of silver, zinc, boron, magnesium, aluminum, vanadium, chromium, manganese, gallium, palladium, osmium and zirconium, to form an alcohol composition containing methanol and ethanol.

In one embodiment, the syngas stream entering the reactor has a stoichiometric ratio of less than 1.9. Preferably, the syngas stream entering the reactor has a stoichiometric ratio of not greater than 1.5. More preferably, the syngas stream entering the reactor has a stoichiometric ratio of not greater than 1.2. In another embodiment, the syngas stream entering the reactor has a stoichiometric ratio of at least 0.1. Preferably, the syngas stream entering the reactor has a stoichiometric ratio of at least 0.5.

In another embodiment, the catalyst comprises from 10 wt % to 70 wt % copper, on an oxide basis.

In yet another embodiment, the catalyst comprises an oxide of zinc. Preferably, the catalyst comprises from 3 wt % to 40 wt % zinc oxide, based on total weight of the catalyst.

In another embodiment of the invention, the catalyst comprises an oxide of aluminum. Preferably, the catalyst comprises from 1 wt. % to 15 wt % of an oxide of aluminum, based on total weight of the catalyst.

In yet another embodiment of the invention, the catalyst comprises 10 wt % to 70 wt % copper, on an oxide weight basis, from 3 wt % 40 wt % zinc oxide, based on total weight of the catalyst, and from 1 wt % to 15 wt % aluminum oxide, based on total weight of the catalyst. Preferably, the copper and zinc are present at a Cu:Zn atomic ratio of from 0.5:1 to 20:1.

In still another embodiment, the catalyst comprises at least one alkali or alkaline earth metal. In one more embodiment, the catalyst comprises at least one alkali metal selected from the group consisting of lithium, sodium, potassium, rubidium, cesium and francium. Preferably, the catalyst comprises from 0.1 wt % to 2 wt % of an alkali or alkaline earth metal, based on total weight of the catalyst.

In a particular embodiment, the catalyst comprises lithium. Alternatively, the catalyst comprises at least one alkaline earth metal selected from the group consisting of calcium, barium, strontium and radium. Preferably, the catalyst comprises calcium.

In another aspect of the invention, the alcohol is contacted with a molecular sieve catalyst to form an olefin product. At least one olefin component of the olefin product can be contacted with a polymer forming catalyst to form a polyolefin product.

According to another aspect of the invention, there is provided a catalyst composition. The composition preferably includes 10 wt % to 70 wt % copper, on an oxide weight basis, 3 wt % 40 wt % zinc oxide, based on total weight of the catalyst, 1 wt % to 15 wt % aluminum oxide, based on total weight of the catalyst; and at least one alkali metal. More preferably, the copper and zinc are present at a Cu:Zn atomic ratio of from 0.5:1 to 20:1.

In one embodiment, the alkali metal is selected from the group consisting of lithium and sodium. Preferably, the alkali metal is lithium.

In another embodiment, the catalyst comprises 15 wt % to 68 wt % copper. Preferably, the catalyst comprises 20 wt % to 65 wt % copper.

In yet another embodiment, the catalyst comprises 4 wt % to 35 wt % zinc oxide. Preferably, the catalyst comprises 5 wt % to 30 wt % zinc oxide.

In still another embodiment, the catalyst comprises 1.5 wt % to 12 wt % aluminum oxide. Preferably, the catalyst comprises 2 wt % to 10 wt % aluminum oxide, more preferably 3 wt % to 8 wt % aluminum oxide, and still more preferably 4 wt % to 6 wt % aluminum oxide.

In another embodiment of the invention, the copper and zinc are present at a Cu:Zn atomic ratio of from 0.7:1 to 15:1. Preferably, the copper and zinc are present at a Cu:Zn atomic ratio of from 0.8:1 to 5:1, more preferably at a Cu:Zn atomic ratio of from 1.5:1 to 2.5:1

In one more embodiment, the alkali metal is selected from the group consisting of lithium and sodium, and the catalyst comprises 0.1 wt % to 2 wt % of the alkali metal.

### BRIEF DESCRIPTION OF THE DRAWING

The attached Figure represents merely one aspect of the invention. The Figure is intended to be viewed as merely one of numerous embodiments within the scope of the overall invention as claimed. Specifically, the Figure is a flow diagram showing conversion of syngas to alcohol product.

### DETAILED DESCRIPTION OF THE INVENTION

### I. Manufacture of Mixed Alcohol and Olefin Product

This invention is directed to a process and catalyst for making an olefin product from a mixed alcohol feed stream. The mixed alcohol stream is made from a synthesis gas (syngas) feed stream. The syngas feed is converted to the mixed alcohol stream using a catalyst comprising copper and at least one oxide component. Upon contacting the catalyst with a desired syngas composition, a preferred mixed alcohol product is formed. Preferably, the syngas composition has a stoichiometric molar ratio of less than 2.

The alcohol product that is formed using this invention contains significant quantities of methanol and ethanol. The product is low in saturates (i.e., paraffins). In general, the alcohol product contains not greater than 2 wt % paraffins, and in preferred embodiments not greater than 1.5 wt % paraffins.

### II. Synthesis Gas Production

The methanol manufacturing process of this invention uses synthesis gas (syngas) as feed. Synthesis gas comprises carbon monoxide and hydrogen. Optionally, carbon dioxide and nitrogen are included.

Synthesis gas can be manufactured from a variety of carbon or hydrocarbon sources. Examples of such sources include biomass, natural gas, C₁-C₅ hydrocarbons, naphtha, heavy petroleum oils, or coke (i.e., coal). Preferably, the hydrocarbon feed stream comprises methane in an amount of at least about 50% by volume, more preferably at least about 70% by volume, most preferably at least about 80% by volume. In one embodiment of this invention natural gas is the preferred hydrocarbon feed source.

Although synthesis gas can be manufactured from a variety of carbon sources, a preferred embodiment of the invention uses natural gas feedstocks comprising methane. The transformation of hydrocarbons into syngas is an endothermic reaction, meaning that heat must be supplied to make the reaction proceed. There are generally two methods of adding heat: (i) indirect heating, generally by burning a fuel and transferring this heat across a metal membrane to the reaction zone, and (ii) in-situ heat generation by adding oxygen to the reformer feed, which results in exothermic oxidation reactions which supply heat for the endothermic reforming reactions. Steam is typically used in method (i), and this process is generally referred to as steam reforming. In method (ii), oxygen is typically added, and this process is generally referred to as oxygen-blown reforming. Various combinations of (i) and (ii) are possible, and are typically referred to as combined reforming.

Oxygen blown reforming can be operated with or without a catalyst. When no catalyst is used, the process is typically referred to as partial oxidation, or POX. The feedstock hydrocarbon and oxygen-containing gas are preheated and react in a burner. When catalyst is used, the oxygen-blown reforming can be further subdivided into two categories. If the feedstock hydrocarbon and oxygen-containing gas are pre-mixed, without reaction, before passing across a catalyst bed, the process is generally referred to as catalytic partial oxidation, or CPOX. When preheated feedstock and oxygen are combined in a burner, where exothermic reactions occur, before passing across a catalyst bed, the process is generally referred to as autothermal reforming, or ATR.

Steam reformers operated with natural gas feedstock produce syngas that is rich in hydrogen, with stoichiometric molar ratios (S_{N}) approaching 3.0. The stoichiometric molar ratio, which accounts for the interconvertability of CO and CO₂, is defined as:

$${S}_{n} = \frac{{H}_{2} - {CO}_{2}}{CO + {CO}_{2}}$$

Oxygen-blown reformers produce syngas that is less rich in hydrogen. These reformers generally have S_{N} values below the theoretical required value for alcohols of 2.0.

The amount of oxygen added to an oxygen-blown reformer depends upon the pressure, feed temperatures, feed compositions and diluent rates, and the desired level of conversion. The total molar flow rate of oxygen added to the reformer, divided by the flow rate of hydrocarbon-based carbon atoms fed to the reformer, will be referred to as the oxygen:carbon ratio.

Steam may be added to an oxygen-blown reformer, either as diluent to the hydrocarbon feedstock, diluent to the oxygen-containing gas, or may be directly injected into a specific portion of the reforming reactor to achieve localized cooling. The total molar flow rate of steam added to the oxygen-blown reformer in any manner, divided by the flow rate of hydrocarbon-based carbon atoms fed to the reformer, will be referred to as the steam:carbon ratio.

In the autothermal reforming of natural gas, steam is added to the feed as a means to reduce or eliminate soot formation, to cool select components of the burner within the ATR, and to reduce the methane content of the syngas at a given temperature. It is desirable to minimize the amount of steam added, so that less energy is required for generation of the steam, and so that the volume of gas passing through the reformer is minimized. ATR reactors generally require steam:carbon ratios of 1.2 to 2.0, although more recent technology allows operation in the 0.4 to 1.2 range. The oxygen:carbon ratio of an ATR ranges between 0.4 to 0.8, preferably between 0.5 and 0.6.

In the partial oxidation of natural gas, the formation of soot is less of a concern because there is no catalyst bed to become fouled or plugged by the soot. Therefore, POX reformers can run with very little or no steam, with steam:carbon ratios between 0 and 0.2. the oxygen:carbon ratio of POX reformers ranges between 0.4 to 0.8, preferably between 0.55 and 0.7.

The catalytic partial oxidation reforming process has not been commercialized to-date for methanol synthesis applications. A CPOX reforming process can theoretically operate with steam:carbon ratios below 0.2, or at any higher level of steam:carbon. A CPOX reformer will generally operate with an oxygen:carbon ratio between 0.4 and 0.8.

Any oxygen-blown reforming reactor will generally reach a product gas that approaches the most thermodynamically stable composition. One skilled in the art can calculate the adiabatic temperature and product syngas composition at thermodynamic equilibrium for any given feed composition, feed temperature, and pressure. The relative amounts of CO and CO₂ in the syngas is determined by the water gas shift (WGS) reaction equilibrium represented by the following:

CO+H₂O [symbol] CO₂+H₂

Increasing the level of steam added to the reformer will shift the reaction to the right, resulting in a greater proportion of CO₂ relative to CO. Higher reaction temperatures shift the equilibrium of the WGS to the left, resulting in reduced proportions of CO₂ relative to CO. It is easily shown that the stoichiometric number for methanol, S_{N}, is not affected by shifting between CO and CO₂ due to the WGS reaction. The CO₂ content of the syngas, however, is affected by the WGS reaction. Low levels of CO₂ in the syngas are favored by low steam:carbon and high temperatures in the reformer.

In the partial oxidation process, the catalyst comprises at least one transition element selected from the group consisting of Ni, Co, Pd, Ru, Rh, Ir, Pt, Os and Fe. Preferably, the catalyst comprises at least one transition element selected from the group consisting of Pd, Pt, and Rh. In another embodiment, preferably the catalyst comprises at least one transition element selected form the group consisting of Ru, Rh, and Ir.

In one embodiment, the partial oxidation catalyst further comprises at least one metal selected from the group consisting of Ti, Zr, Hf. Y, Th, U, Zn, Cd, B, Al, Ti, Si, Sn, Pb, P, Sb, Bi, Mg, Ca, Sr, Ba, Ga, V, and Sc. Also, optionally included in the partial oxidation catalyst is at least one rare earth element selected from the group consisting of La, Ce, Pr, Nd, Pm, Sm, Eu, Gd, Th, Dy, Ho, Er, Tm, Yb and Lu.

In another embodiment the catalyst employed in the process comprises a wide range of catalytically active components, for example Pd, Pt, Rh, Ir, Os, Ru, Ni, Cr, Co, Ce, La and mixtures thereof. Materials not normally considered to be catalytically active may also be employed as catalysts, for example refractory oxides such as cordierite, mullite, mullite aluminum titanate, zirconia spinels and alumina.

In yet another embodiment, the catalyst is comprised of metals selected from those having atomic number 21 to 29, 40 to 47 and 72 to 79, the metals Sc, Ti V, Cr, Mn, Fe, Co, Ni, Cu, Zr, Nb, Mo, Tc, Ru, Rhh, Pd, Ag, Hf, Ta, W, Re, Os Ir, Pt, and Au. The preferred metals are those in Group 8 of the Periodic Table of the Elements, that is Fe, Os, Co, Re, Ir, Pd, Pt, Ni, and Ru.

In another embodiment, the partial oxidation catalyst comprises at least one transition or non-transition metal deposited on a monolith support. The monolith supports are preferably impregnated with a noble metal such as Pt, Pd or Rh, or other transition metals such as Ni, Co, Cr and the like. Desirably, these monolith supports are prepared from solid refractory or ceramic materials such as alumina, zirconia, magnesia, ceria, silica, titania, mixtures thereof, and the like. Mixed refractory oxides, that is refractory oxides comprising at least two cations, may also be employed as carrier materials for the catalyst.

The partial oxidation process is operable over a wide range of pressures. For applications on a commercial scale, elevated pressures, that is pressures significantly above atmospheric pressure, are preferred. In one embodiment, the partial oxidation process is operated at pressures of greater than atmospheric up to about 150 bars. Preferably, the partial oxidation process is operated at a pressure in the range of from about 2 bars to about 125 bars, more preferably from about 5 bars to about 100 bars.

The partial oxidation process is also operable over a wide range of temperatures. At commercial scale, the feed is preferably contacted with the catalyst at high temperatures. In one embodiment, the feed mixture is contacted with the catalyst at a temperature in excess of 600° C. Preferably, the feed mixture is contacted with the catalyst at a temperature in the range of from about 600° C. to about 1,700° C., more preferably from about 800° C. to about 1,600° C. The feed mixture is preferably preheated prior to contacting the catalyst.

### III. Syngas Feed to the Alcohol Synthesis Process

Synthesis gas (syngas) is used as a feedstock to form a mixed alcohol product. In one embodiment of the invention, the synthesis gas feed (including any recycle syngas recovered from the process itself as well as fresh syngas) has a molar ratio of hydrogen (H₂) to carbon oxides (CO+CO₂) in the range of from about 0.5:1 to about 20:1, preferably in the range of from about 1:1 to about 10:1. In another embodiment, the synthesis gas has a molar ratio of hydrogen (H₂) to carbon monoxide (CO) of at least 2:1. Carbon dioxide is optionally present in an amount of not greater than 50% by weight, based on total weight of the synthesis gas, and preferably less than 20% by weight, more preferably less than 10% by weight.

The stoichiometric molar ratio (i.e., a molar ratio of (H₂—CO₂)/(CO+CO₂)) of the syngas should be sufficiently high so as maintain a high yield of methanol and ethanol, but not so high as to reduce the volume productivity of both the methanol and ethanol. In one embodiment, the synthesis gas fed to the alcohol synthesis reactor has a stoichiometric molar ratio of less than 2. Preferably, the syngas stream that enters the reactor has a stoichiometric ratio of less than 1.9, more preferably not greater than 1.5, and most preferably not greater than 1.2. It is also preferred that the syngas stream that enters the reactor have a stoichiometric ratio of at least 0.1, more preferably at least 0.5.

In some embodiments of the invention, at least a portion of the syngas that is not reacted in the alcohol conversion reactor is recovered and recycled as feed. In one embodiment, it is preferred that the stoichiometric molar ratio of the syngas from the syngas unit be in a range of from 1.5 to 1.9, prior to mixing with recycle syngas recovered from the alcohol conversion unit. When the recycle syngas is combined with syngas from a syngas unit, the stoichiometric ratio of the combined gas (recycle syngas plus syngas from the syngas unit) is reduced. Increasing the ratio of recycle gas to make-up gas increases the alcohol production rate for a given amount of syngas. Preferably, recycle syngas is added to syngas from a syngas unit (make-up gas) at a ratio of recycle gas to make-up gas of at least 1.0, more preferably at least 2.0. It is also preferred that the ratio of recycle gas to makeup gas is not greater than 5.0, more preferably not greater than 4.0.

### IV. Converting Syngas to Mixed Alcohol

The catalyst used in converting the syngas to the alcohol product stream in this invention includes copper and an oxide of at least one element selected from the group consisting of silver, zinc, boron, magnesium, aluminum, vanadium, chromium, manganese, gallium, palladium, osmium and zirconium.

In a particularly preferred embodiment of the invention, the catalyst comprises copper and an oxide of at least one element selected from the group consisting of zinc, magnesium, aluminum, chromium, and zirconium. Most preferably, the catalyst comprises copper and zinc oxide. Even more preferably, the catalyst comprises copper, zinc oxide and aluminum oxide.

Preferably, the catalyst used in this invention comprises from about 10 wt % to about 70 wt % copper, on an oxide weight basis. More preferably, the catalyst used in this invention comprises from 15 wt % to 68 wt % copper, and most preferably from 20 wt % to 65 wt % copper, on an oxide weight basis.

In another embodiment of the invention, the catalyst comprises zinc oxide. Preferably, the zinc oxide containing catalyst comprises from about 3 wt % to about 40 wt % zinc oxide, based on total weight of the catalyst. More preferably, the zinc oxide containing catalyst comprises from 4 wt % to 35 wt % zinc oxide, and most preferably from 5 wt % to 30 wt % zinc oxide.

In embodiments in which copper and zinc oxide are both present in the catalyst, the ratio of copper, on an oxide weight basis, to zinc oxide comprises copper and zinc oxide in a Cu:Zn atomic ratio of from about 0.5:1 to about 20:1, preferably from 0.7:1 to 15:1, more preferably from 0.8:1 to 5:1, and most preferably from 1.5:1 to 2.5:1.

In yet another embodiment, the catalyst comprises aluminum oxide. Preferably, the aluminum oxide containing catalyst comprises from about 1 wt % to about 15 wt % aluminum oxide, based on total weight of the catalyst. More preferably, the aluminum oxide containing catalyst comprises from 1.5 wt % to 12 wt % aluminum oxide, still more preferably from 2 wt % to 10 wt % aluminum oxide, even preferably from 3 wt % to 8 wt % aluminum oxide, and most preferably from 4 wt % to 6 wt % aluminum oxide.

In another embodiment of the invention, the catalyst comprises at least one alkali or alkaline earth metal. Such an embodiment includes at least one alkali metal selected from the group consisting of lithium, sodium, potassium, rubidium, cesium and francium. Another embodiment includes at least one alkaline earth metal selected from the group consisting of calcium, barium, strontium and radium.

In a particular embodiment, the catalyst comprises from 0.1 wt % to 2 wt % of an alkali or alkaline earth metal, based on total weight of the catalyst. Preferably, the catalyst comprises lithium, sodium or both as the alkali metal. More preferably, the catalyst comprises lithium as the alkali metal. Alternatively, the catalyst comprises calcium as the alkaline earth metal.

The conversion of syngas to alcohol product can be accomplished over a wide range of temperatures. Lower temperature ranges are preferred. In one embodiment, the syngas is contacted with the catalysts at a temperature in the range of from about 150° C. to about 450° C., preferably in a range of from about 175° C. to about 350° C., more preferably in a range of from about 200° C. to about 325° C., and most preferably from about 260° C. to about 300° C.

The syngas can also be converted to alcohol product over a wide range of pressures. In one embodiment, the syngas is contacted with the catalyst at a pressure in the range of from about 15 atmospheres to about 150 atmospheres, preferably in a range of from about 25 atmospheres to about 125 atmospheres, more preferably in a range of from about 50 atmospheres to about 125 atmospheres.

Gas hourly space velocities in converting the syngas to alcohol product can vary depending upon the type of reactor that is used. In one embodiment, gas hourly space velocity of flow of gas through the catalyst bed is in the range of from about 50 hr⁻¹ to about 50,000 hr⁻¹. Preferably, gas hourly space velocity of flow of gas through the catalyst bed is in the range of from about 250 hr⁻¹ to about 25,000 hr⁻¹, more preferably from about 500 hr⁶⁻¹ to about 10,000 hr⁻¹.

The process can be carried out in one or more than one reactor, in series or parallel. Preferably, the process is carried out in a single reactor.

The alcohol that is formed in the process preferably contains at least 70 wt % methanol and at least 4 wt % ethanol, on a total dry weight basis. In one embodiment, the alcohol contains propanol, preferably at least 1 wt %, on a total dry weight basis. Preferably, the alcohol contains less than 2 wt % hydrocarbons (i.e., paraffins, such as methane, ethane, etc.).

### V. Recovery and Further Processing of Methanol Product

The alcohol product can be used “as is,” or it can be further processed if desired. Processing can be accomplished using any type of separation or recovery means for recovering the methanol and ethanol components. Examples of such recovery means include distillation, selective condensation, and selective adsorption. Process conditions, e.g., temperatures and pressures, can vary according to the particular alcohol composition desired. It is particularly desirable to minimize the amount of water and light boiling point components in the alcohol composition, but without substantially reducing the amount of methanol and ethanol present.

In one embodiment, the separated and recovered alcohol product is sent to a let down vessel so as to reduce the pressure to about atmospheric or slightly higher. This let down in pressure allows undesirable light boiling point components to be removed from the alcohol composition as a vapor. The vapor is desirably of sufficient quality to use a fuel.

In another embodiment, the separated recovered alcohol product is sent from the alcohol synthesizing unit or vessel to a distillation system. The distillation system contains one or more distillation columns which are used to further separate the desired methanol and ethanol composition from water and hydrocarbon by-product streams. Desirably, the methanol and ethanol composition that is separated from the crude alcohol composition comprises a majority of the methanol and ethanol contained in the alcohol product prior to separation.

In one embodiment, the distillation system includes a step of treating the recovered alcohol product stream being distilled so as to remove or neutralize acids in the stream. Preferably, a base is added in the system that is effective in neutralizing organic acids that are found in the alcohol stream. Conventional base compounds can be used. Examples of base compounds include alkali metal hydroxide or carbonate compounds, and amine or ammonium hydroxide compounds. In one particular embodiment, about 20 ppm to about 120 ppm w/w of a base composition, calculated as stoichiometrically equivalent NaOH, is added, preferably about 25 ppm to about 100 ppm w/w of a base composition, calculated as stoichiometrically equivalent NaOH, is added.

Examples of distillation systems include the use of single and two column distillation columns. Preferably, the single columns operate to remove volatiles in the overhead, mixed methanol and ethanol product at a high level, fusel oil as vapor above the feed and/or as liquid below the feed, and water as a bottoms stream.

In one embodiment of a two column system, the first column is a “topping column” from which volatiles are taken overhead and methanol and ethanol liquid as bottoms. The second is a “rectifying column” from which methanol and ethanol product is taken as an overhead stream or at a high level, and water is removed as a bottoms stream. In this embodiment, the rectifying column includes at least one off-take for fusel oil as vapor above the feed and/or as liquid below the feed.

In another embodiment of a two column system, the first column is a water-extractive column in which there is a water feed introduced at a level above the crude alcohol feed level. It is desirable to feed sufficient water to produce a bottoms liquid containing over 40% w/w water, preferably 40% to 60% w/w water, and more preferably 80% to 95% w/w water. This column optionally includes one or more direct fusel oil side off-takes.

In yet another embodiment, the distillation system is one in which an aqueous, semi-crude alcohol is taken as liquid above the feed in a single or rectifying column. The semi-crude alcohol is passed to a rectifying column, from which methanol and ethanol product is taken overhead or at a high level.

Preferably, a water or aqueous methanol and ethanol stream is taken as a bottoms stream.

Alternatively, undesirable by-products are removed from the separated methanol and ethanol stream from the alcohol synthesis reactor by adsorption. In such a system, other components such as fusel oil can be recovered by regenerating the adsorbent.

### VI. Converting the Alcohol Composition to Olefins

### A. General Process Description

In one embodiment of the invention, the alcohol composition is converted to olefins by contacting the alcohol composition with an olefin forming catalyst to form the olefin product. The olefin product is recovered, and water, which forms during the conversion of the oxygenates in the methanol to olefins, is removed. After removing the water, the olefins are separated into individual olefin streams, and each individual olefin stream is available for further processing.

### B. Description of Olefin Forming Catalyst

Any catalyst capable of converting oxygenate to olefin can be used in this invention. Molecular sieve catalysts are preferred. Examples of such catalysts include zeolite as well as non-zeolite molecular sieves, and are of the large, medium or small pore type. Non-limiting examples of these molecular sieves are the small pore molecular sieves, AEI, AFT, APC, ATN, ATT, ATV, AWW, BIK, CAS, CHA, CHI, DAC, DDR, EDI, ERI, GOO, KFI, LEV, LOV, LTA, MON, PAU, PHI, RHO, ROG, THO, and substituted forms thereof; the medium pore molecular sieves, AFO, AEL, EUO, HEU, FER, MEL, MFI, MTW, MTT, TON, and substituted forms thereof; and the large pore molecular sieves, EMT, FAU, and substituted forms thereof. Other molecular sieves include ANA, BEA, CFI, CLO, DON, GIS, LTL, MER, MOR, MWW and SOD. Non-limiting examples of the preferred molecular sieves, particularly for converting an oxygenate containing feedstock into olefin(s), include AEL, AFY, BEA, CHA, EDI, FAU, FER, GIS, LTA, LTL, MER, MFI, MOR, MTT, MWW, TAM and TON. In one preferred embodiment, the molecular sieve of the invention has an AEI topology or a CHA topology, or a combination thereof, most preferably a CHA topology.

In one embodiment, aluminophosphate (ALPO) molecular sieves, silicoaluminophosphate (SAPO) molecular sieves or a combination thereof is used. Preferred molecular sieves are SAPO molecular sieves, and metal substituted SAPO molecular sieves. In an embodiment, the metal is an alkali metal of Group IA of the Periodic Table of Elements, an alkaline earth-metal of Group IIA of the Periodic Table of Elements, a rare earth metal of Group IIIB, including the Lanthamides: lanthanum, cerium, praseodymium, neodymium, samarium, europium, gadolinium, terbium, dysprosium, holmium, erbium, thulium, ytterbium and lutetium; and scandium or yttrium of the Periodic Table of Elements, a transition metal of Groups IVB, VB, VIIB, VIIB, VIIIB, and IB of the Periodic Table of Elements, or mixtures of any of these metal species. In one preferred embodiment, the metal is selected from the group consisting of Co, Cr, Cu, Fe, Ga, Ge, Mg, Mn, Ni, Sn, Ti, Zn and Zr, and mixtures thereof. In another preferred embodiment, these metal atoms discussed above are inserted into the framework of a molecular sieve through a tetrahedral unit, such as [MeO₂], and carry a net charge depending on the valence state of the metal substituent. For example, in one embodiment, when the metal substituent has a valence state of +2, +3, +4, +5, or +6, the net charge of the tetrahedral unit is between −2 and +2.

Non-limiting examples of SAPO and ALPO molecular sieves used in the invention include one or a combination of SAPO-5, SAPO-8, SAPO-11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44 (U.S. Pat. No. 6,162,415), SAPO-47, SAPO-56, ALPO-5, ALPO-11, ALPO-18, ALPO-31, ALPO-34, ALPO-36, ALPO-37, ALPO-46, and metal containing molecular sieves thereof. The more preferred zeolite-type molecular sieves include one or a combination of SAPO-18, SAPO-34, SAPO-35, SAPO-44, SAPO-56, ALPO-18 and ALPO-34, even more preferably one or a combination of SAPO-18, SAPO-34, ALPO-34 and ALPO-18, and metal containing molecular sieves thereof, and most preferably one or a combination of SAPO-34 and ALPO-18, and metal containing molecular sieves thereof.

In an embodiment, the molecular sieve is an intergrowth material having two or more distinct phases of crystalline structures within one molecular sieve composition. In particular, intergrowth molecular sieves are described in the U.S. patent application Ser. No. 09/924,016 filed Aug. 7, 2001 and PCT WO 98/15496 published Apr. 16, 1998, both of which are herein fully incorporated by reference. In another embodiment, the molecular sieve comprises at least one intergrown phase of AEI and CHA framework-types. For example, SAPO-18, ALPO-18 and RUW-18 have an AEI framework-type, and SAPO-34 has a CHA framework-type.

The molecular sieves are made or formulated into catalysts by combining the synthesized molecular sieves with a binder and/or a matrix material to form a molecular sieve catalyst composition or a formulated molecular sieve catalyst composition. This formulated molecular sieve catalyst composition is formed into useful shape and sized particles by conventional techniques such as spray drying, pelletizing, extrusion, and the like.

### C. General Conditions for Converting Alcohol to Olefins

According to the reaction process of this invention, the mixed alcohol stream is contacted with olefin forming catalyst to form an olefin product, particularly ethylene and propylene. The process for converting the oxygenate feedstock is, preferably, a continuous fluidized bed process, and most preferably a continuous high velocity fluidized bed process.

The reaction processes can take place in a variety of catalytic reactors such as hybrid reactors that have a dense bed or fixed bed reaction zones and/or fast fluidized bed reaction zones coupled together, circulating fluidized bed reactors, riser reactors, and the like. Suitable conventional reactor types are described in for example U.S. Pat. No. 4,076,796, U.S. Pat. No. 6,287,522 (dual riser), and Fluidization Engineering, D. Kunii and O. Levenspiel, Robert E. Krieger Publishing Company, New York, N.Y. 1977.

One preferred reactor type is a riser reactor. These types of reactors are generally described in Riser Reactor, Fluidization and Fluid-Particle Systems, pages 48 to 59, F. A. Zenz and D. F. Othmo, Reinhold Publishing Corporation, New York, 1960, and U.S. Pat. No. 6,166,282 (fast-fluidized bed reactor).

In one embodiment of the invention, a fluidized bed process or high velocity fluidized bed process includes a reactor system, catalyst separation system, and a regeneration system. The reactor system preferably is a fluid bed reactor system. In one embodiment, the fluid bed reactor system has a first reaction zone within one or more riser reactors, and a second reaction zone within at least one catalyst separation vessel, preferably comprising one or more cyclones. In one embodiment, one or more riser reactors and catalyst separation vessel is contained within a single reactor vessel.

The average reaction temperature employed in the conversion process, specifically within the reactor, is of from about 250° C. to about 800° C. Preferably the average reaction temperature within the reactor is from about 250° C. to about 750° C.; more preferably, from about 300° C. to about 650° C.; yet more preferably from about 350° C. to about 600° C.; and most preferably from about 400° C. to about 500° C.

The pressure employed in the conversion process, specifically within the reactor, is not critical. The reaction pressure is based on the partial pressure of the feedstock exclusive of any diluent therein. Typically the reaction pressure employed in the process is in the range of from about 0.1 kPaa to about 5 MPaa, preferably from about 5 kPaa to about 1 MPaa, and most preferably from about 20 kPaa to about 500 kPaa.

The weight hourly space velocity (WHSV), defined as the total weight of the feedstock excluding any diluents to the reaction zone per hour per weight of molecular sieve in the molecular sieve catalyst composition in the reaction zone, is maintained at a level sufficient to keep the catalyst composition in a fluidized state within a reactor. Typically, the WHSV ranges from about 1 hr⁻¹ to about 5000 hr⁻¹, preferably from about 2 hr⁻¹ to about 3000 hr⁻¹, more preferably from about 5 hr⁻¹ to about 1500 hr⁻¹, and most preferably from about 10 hr⁻¹ to about 1000 hr⁻¹. In one preferred embodiment, the WHSV is greater than 20 hr⁻¹, preferably the WHSV for conversion of a feedstock containing methanol and dimethyl ether is in the range of from about 20 hr⁻¹ to about 300 hr⁻¹.

The superficial gas velocity (SGV) of the feedstock including diluent and reaction products within the reactor is preferably sufficient to fluidize the molecular sieve catalyst composition within a reaction zone of the reactor. The SGV in the process, particularly within the reactor system, more particularly within a riser reactor, is at least 0.1 meter per second (m/sec), preferably greater than 0.5 m/sec, more preferably greater than 1 m/sec, even more preferably greater than 2 m/sec, yet even more preferably greater than 3 m/sec, and most preferably greater than 4 m/sec.

### VII. Olefin Product Recovery and Use

In one embodiment, olefin product and other gases are withdrawn from the reactor and are passed through a recovery system. Any conventional recovery system, technique and/or sequence useful in separating olefin(s) and purifying olefin(s) from other gaseous components can be used in this invention. Examples of recovery systems include one or more or a combination of various separation, fractionation and/or distillation towers, columns, and splitters, and other associated equipment; for example, various condensers, heat exchangers, refrigeration systems or chill trains, compressors, knock-out drums or pots, pumps, and the like.

Non-limiting examples of distillation towers, columns, splitters or trains used alone or in combination include one or more of a demethanizer, preferably a high temperature demethanizer, a deethanizer, a depropanizer, preferably a wet depropanizer, a wash tower often referred to as a caustic wash tower and/or quench tower, absorbers, adsorbers, membranes, ethylene (C₂) splitter, propylene (C₃) splitter, butene (C₄) splitter, and the like.

Generally accompanying most recovery systems is the production, generation or accumulation of additional products, by-products and/or contaminants along with the preferred prime products. The preferred prime products, the light olefins, such as ethylene and propylene, are typically purified for use in derivative manufacturing processes such as polymerization processes.

The ethylene and propylene streams produced and recovered according to this invention can be polymerized to form plastic compositions, e.g., polyolefins, particularly polyethylene and polypropylene. Any process capable of forming polyethylene or polypropylene can be used. Catalytic processes are preferred. Particularly preferred are metallocene, Ziegler/Natta, aluminum oxide and acid catalytic systems. In general, these methods involve contacting the ethylene or propylene product with a polyolefin-forming catalyst at a pressure and temperature effective to form the polyolefin product.

In one embodiment of this invention, the ethylene or propylene product is contacted with a metallocene catalyst to form a polyolefin. Desirably, the polyolefin forming process is carried out at a temperature ranging between about 50° C. and about 320° C. The reaction can be carried out at low, medium or high pressure, being anywhere within the range of about 1 bar to about 3200 bar. For processes carried out in solution, an inert diluent can be used. In this type of operation, it is desirable that the pressure be at a range of from about 10 bar to about 150 bar, and preferably at a temperature range of from about 120° C. to about 250° C. For gas phase processes, it is preferred that the temperature generally be within a range of about 60° C. to 120° C., and that the operating pressure be from about 5 bar to about 50 bar.

In addition to polyolefins, numerous other olefin derivatives may be formed from the ethylene, propylene and C₄+ olefins, particularly butylene, separated according to this invention. The olefins separated according to this invention can also be used in the manufacture of such compounds as aldehydes, acids such as C₂-C₁₃ mono carboxylic acids, alcohols such as C₂-C₁₂ mono alcohols, esters made from the C₂-C₁₂ mono carboxylic acids and the C₂-C₁₂ mono alcohols, linear alpha olefins, vinyl acetate, ethylene dicholoride and vinyl chloride, ethylbenzene, ethylene oxide, cumene, acrolein, allyl chloride, propylene oxide, acrylic acid, ethylene-propylene rubbers, and acrylonitrile, and trimers and dimers of ethylene and propylene. The C₄+ olefins, butylene in particular, are particularly suited for the manufacture of aldehydes, acids, alcohols, esters made from C₅-C₁₃ mono carboxylic acids and C₅-C₁₃ mono alcohols and linear alpha olefins.

### EXAMPLES

### Example 1

This example describes the preparation of an alcohol conversion catalyst using the co-precipitation procedure described below, and having the following composition: 60% CuO/30% ZnO/10% A 1203

The following solutions were prepared:

2 M Cu: 36.4 g Cu(NO₃)₂*3H₂O were dissolved in 75 cc d.i. H₂O

2 M Zn: 22.0 g Zn(NO₃)₂*6H₂O were dissolved in 37 cc d.i. H₂O

2 M Al: 14.8 g Al(NO₃)₃*9H₂O were dissolved in 20 cc d.i. H₂O

2 M Na₂CO₃: 93.0 g Na₂CO₃*H₂O were dissolved in 375 cc d.i. H₂O

A 2 liter flask was filled with 500 ml d.i. H₂O. The water was heated to 70° C. and stirred. The Cu, Zn and Al solutions were mixed to form solution 1. The Na₂CO₃ solution was labeled as solution 2. While stirring the water and maintaining its temperature at 70° C., solutions 1 and 2 were added simultaneously to the water with pH controlled during addition at 7.0. The reaction was considered complete when all of solution 1 had been added. The mixture was then kept at temperature and under stirring for another hour. The stirring was stopped and the mixture was cooled to room temperature. The precipitate was then filtered and thoroughly washed with warm d.i. H₂O (ca. 1500-2000 cc H₂O). The precipitate was finally dried overnight at 85° C.

The precipitate was calcined under air according to the following schedule:

1) 2 hours ramp from room temperature to 150° C.

2) 0.5 hour at 150° C.

3) 2 hours ramp from 150° C. to 350° C.

4) 3 hours at 350° C., then heating stopped

5) cool down to room temperature

A portion of the catalyst was loaded into a reactor and tested for syngas conversion under the following conditions: T=250° C., P=750 psia, GHSV=5000, feed composition (molar basis): 60% H₂, 20% CO, 5% CO₂, 15% N₂. Average conversions and selectivities over a 20 hour period were run. The ethanol and methanol selectivities were 0.4 and 98.0 wt %, respectively. The results are shown in Table 1.

### Example 2

A portion of the catalyst of Example 1 was loaded into a reactor and tested for syngas conversion under the following conditions: T=287.5° C., P=1008 psia, GHSV=4999, feed composition (molar basis): 44% H₂, 44% CO, 2% CO₂, 10% N₂. Average conversions and selectivities over a 20 hour period were run. The ethanol and methanol selectivities were 4.2 and 81.3 wt %, respectively. The results are shown in Table 1. As seen from Table 1, this example shows a significant increase in ethanol content relative to Example 1. A higher temperature and lower H₂ content syngas were used compared to Example 1.

### Example 3

Catalyst was prepared similar to the procedure in Example 1, except that proportions were adjusted to provide the following catalyst composition: 60% CuO/35% ZnO/5% Al₂O₃.

A portion of the catalyst was loaded into a reactor and tested for syngas conversion under the following conditions: T=287.3° C., P=1000 psia, GHSV=5005, feed composition: 44% H₂, 44% CO, 2% CO₂, 10% N₂. Average conversions and selectivities over a 20 hour period were run. The ethanol and methanol selectivities were 4.9 and 81.9 wt %, respectively. The results are shown in Table 1. The increase of ethanol in this example compared to Example 2 is considered significant. The lower Al₂O₃ content of the catalyst in this example is preferred over the higher Al₂O₃ content in Example 2.

### Example 4

Catalyst was prepared similar to the procedure in Example 3, except that 0.05 wt % lithium was added.

A portion of the catalyst was loaded into a reactor and tested for syngas conversion under the following conditions: T=278.3° C., P=1001 psia, GHSV=5001, feed composition: 44% H₂, 44% CO, 2% CO₂, 10% N₂. Average conversions and selectivities over a 20 hour period were run. The ethanol and methanol selectivities were 5.6 and 80.5 wt %, respectively. The results are shown in Table 1. The increase of ethanol in this example compared to Example 3 is considered significant. The use of Li in the catalyst is also preferred.

TABLE 1

| | Example number | | | |
|---|---|---|---|---|
| | 1 | 2 | 3 | 4 |
| Catalyst | 10% AI₂O₃ | 10% AI₂O₃ | 5% AI₂O₃ | 5% AI₂O₃ |
| | Cu/Zn/Al | CU/Zn/Al | Cu/Zn/Al | Cu/Zn/Al |
| | | | | WITH 0.5% Li |
| Catalyst comp (wt % oxide) | 60/30/10 | 60/30/5 | 60/35/5 | 60/35/5 |
| Feed Comp - H₂/CO/CO₂/N₂ (molar) | 60/20/5/15 | 44/44/2/10 | 44/44/2/10 | 44/44/2/10 |
| Stoichiometric no., Sn | 2.2 | 0.9 | 0.9 | 0.9 |
| GHSV | 5000 | 4999 | 5005 | 5001 |
| Temp, C. | 250 | 287.5 | 287.3 | 278.3 |
| Press, psia | 750 | 1008 | 1000 | 1001 |
| Conversions | | | | |
| H₂ | | 55.98% | 41.81% | 44.90% |
| COₓ | 45 | 28.44% | 20.62% | 23.66% |
| Selectivities (normalized, wt. basis) | | | | |
| MeOH | 98.93% | 81.26% | 81.87% | 80.47% |
| EtOH | 0.38% | 4.17% | 4.90% | 5.59% |
| PrOH | 0.15% | 1.93% | 2.15% | 2.57% |
| BuOH | 0.18% | 1.57% | 1.73% | 2.05% |
| Hydrocarbons | 0.29% | 1.35% | 0.98% | 0.86% |
| DME | 0.00% | 1.91% | 1.86% | 1.07% |
| Methyl formate | 0.00% | 1.05% | 0.98% | 1.01% |
| Ethanal | 0.02% | 0.68% | 0.25% | 0.24% |
| Acetone | 0.00% | 0.04% | 0.02% | 0.02% |
| Methyl acetate | 0.05% | 1.47% | 1.31% | 1.49% |
| Propanal | 0.00% | 0.36% | 0.11% | 0.11% |
| Other oxygenates | 0.00% | 4.21% | 3.82% | 4.53% |

Table 1 shows that a substantial increase in ethanol content is achieved when using a catalyst comprising copper and at least one oxide component as a syngas conversion catalyst and supplying a syngas feed stream having a stoichiometric molar ratio of less than 2.

### Example 5

A computer simulation using an equilibrium reaction model of a partial oxidation reformer was run to show how the syngas composition (Sn) changes when gas prepared from an oxygen-blown reformer (with sub-stoichiometric Sn value) is mixed with recycle syngas from an alcohol synthesis reactor, and the effects of Sn number using a catalyst composition as that of Examples 2, 3, or 4. The Sn value of the blend of fresh and recycle syngas is less than the fresh syngas. Using an Sn value of less than 2 to contact the catalyst is shown to substantially increase ethanol yield.

The flow scheme used in the computer simulation follows that shown in the Figure. According to the Figure, stream S1, a combination of natural gas and oxygen, is sent to a partial oxidation reformer R1, which produces a syngas stream S2. Stream S2 is sent to a separator F1 in which a water stream S3 is separated out as a bottoms stream. A syngas stream S4 is sent to a compressor C1. Stream S5 is recovered from the compressor C1, and is ultimately sent on to an alcohol synthesis reactor R2 as stream S6. An alcohol product stream S7 is recovered from the reactor R2 and send to a separator F2. An overhead stream S9 from the separator F2 is recovered, with part of the stream being recycled as recycle stream S10 to mix with stream S5, and part of the overhead stream is sent to other processes as stream S11.

Bottoms stream S8 from separator F2 is sent to separator F3, where alcohol product is recovered as bottoms stream S13. Overhead stream S12 from separator F3 is recovered and also recycled to mix with stream S5.

The partial oxidation reformer uses no steam and was set to operate at an O₂:C ratio of 0.59. The conversion of natural gas is 95.6%, with stream S4 having a stoichiometric number of 1.77. The recycle gas flow rate is 1.6 times the rate of stream S4, giving a stoichiometric number for stream S6 of 0.73. Additional stream details are shown in Table 2.

TABLE 2

| | | Stream Name | | | | | | | | |
|---|---|---|---|---|---|---|---|---|---|---|
| | | Stream Description | | | | | | | | |
| | | S1 | S2 | S3 | S4 | S6 | S7 | S10 | S11 | S13 |
| Phase | | Vapor | Vapor | Vapor | Vapor | Vapor | Vapor | Vapor | Vapor | Mixed |
| Temperature | C. | 630.00 | 1075.06 | 45.00 | 45.00 | 66.23 | 285.00 | 45.00 | 45.00 | 51.87 |
| Pressure | BAR | 45.000 | 45.000 | 45.000 | 45.000 | 90.000 | 90.000 | 90.000 | 90.000 | 90.000 |
| Flowrate | KG-MOL/ | 2130.00 | 3489.909 | 584.077 | 2905.832 | 8134.176 | 6445.543 | 250.000 | 5158.496 | 967.199 |
| | HR | | | | | | | | | |
| Composition | | | | | | | | | | |
| METHANE | | 0.46948 | 0.01577 | 0.00000 | 0.01894 | 0.17767 | 0.22600 | 0.26628 | 0.26628 | 0.00000 |
| H₂ | | 0.00000 | 0.54432 | 0.00000 | 0.65373 | 0.44131 | 0.27490 | 0.32734 | 0.32734 | 0.00000 |
| H₂O | | 0.28169 | 0.16914 | 1.00000 | 0.00213 | 0.00144 | 0.02934 | 0.00107 | 0.00107 | 0.18959 |
| CO | | 0.00000 | 0.23501 | 0.00000 | 0.28225 | 0.28802 | 0.24785 | 0.29423 | 0.29423 | 0.00000 |
| CO₂ | | 0.00000 | 0.03575 | 0.00000 | 0.04294 | 0.08483 | 0.09159 | 0.10047 | 0.10047 | 0.00000 |
| METHANOL | | 0.00000 | 0.00000 | 0.00000 | 0.00000 | 0.00648 | 0.12197 | 0.01022 | 0.01022 | 0.75570 |
| O₂ | | 0.24883 | 0.00000 | 0.00000 | 0.00000 | 0.00000 | 0.00000 | 0.00000 | 0.00000 | 0.00000 |
| ETHANOL | | 0.00000 | 0.00000 | 0.00000 | 0.00000 | 0.00019 | 0.00536 | 0.00030 | 0.00030 | 0.03405 |
| 1-PROPANOL | | 0.00000 | 0.00000 | 0.00000 | 0.00000 | 0.00006 | 0.00318 | 0.00010 | 0.00010 | 0.02066 |

Table 2 shows that a substantial amount of ethanol is produced using a syngas feed stream having a stoichiometric molar ratio of less than 2.

### Example 6

A computer simulation similar to that of Example 5 was repeated, except using an autothermal reformer with a steam:carbon ratio of 0.6 and an O₂:C ratio of 0.53. The conversion of natural gas is 94.5%, with stream S4 having a stoichiometric number of 1.88. The recycle gas flow rate is 1.8 times the rate of stream S4, giving a stoichiometric number for stream S6 of 0.96. Additional stream details are shown in Table 3.

Table 3 shows that a substantial amount of ethanol is produced using a syngas feed stream having a stoichiometric molar ratio of less than 2.

The principles and modes of operation of this invention have been described above with reference to various exemplary and preferred embodiments. As understood by those of skill in the art, the overall invention, as defined by the claims, encompasses other preferred embodiments not specifically enumerated herein.

The invention is further illustrated but not limited by the following embodiments. This invention further relates to:

### Embodiment 1

A process for producing alcohol from syngas, comprising:

sending a syngas stream to an alcohol synthesis reactor, wherein the syngas stream entering the reactor has a stoichiometric molar ratio of less than 2; and

contacting the syngas in the alcohol synthesis reactor with a catalyst comprising copper and an oxide of at least one element selected from the group consisting of silver, zinc, boron, magnesium, aluminum, vanadium, chromium, manganese, gallium, palladium, osmium and zirconium, to form an alcohol composition containing methanol and ethanol.

### Embodiment 2

The process of embodiment 1, wherein the syngas stream entering the reactor has a stoichiometric ratio of less than 1.9.

TABLE 3

| | | Stream Name | | | | | | | | |
|---|---|---|---|---|---|---|---|---|---|---|
| | | Stream Description | | | | | | | | |
| | | S1 | S2 | S3 | S4 | S6 | S7 | S10 | S11 | S13 |
| Phase | | Vapor | Vapor | Vapor | Vapor | Vapor | Vapor | Vapor | Vapor | Mixed |
| Temperature | C. | 400.00 | 1233.66 | 45.00 | 45.00 | 57.27 | 285.00 | 45.00 | 45.00 | 51.64 |
| Pressure | BAR | 60.000 | 60.000 | 60.000 | 60.000 | 90.000 | 90.000 | 90.000 | 90.000 | 90.000 |
| Flowrate | KG-MOL/ | 1590.000 | 2911.436 | 083.213 | 2728.223 | 7234.172 | 5589.489 | 250.000 | 4443.689 | 833.541 |
| | HR | | | | | | | | | |
| Composition | | | | | | | | | | |
| METHANE | | 0.62893 | 0.01521 | 0.00000 | 0.01623 | 0.11625 | 0.15045 | 0.17691 | 0.17691 | 0.00000 |
| H₂ | | 0.00000 | 0.59210 | 0.00000 | 0.63186 | 0.41148 | 0.23674 | 0.28156 | 0.28156 | 0.00000 |
| H₂O | | 0.00000 | 0.06443 | 1.00000 | 0.00160 | 0.00126 | 0.01659 | 0.00107 | 0.00107 | 0.10525 |
| CO | | 0.00000 | 0.31565 | 0.00000 | 0.33685 | 0.39436 | 0.38537 | 0.43350 | 0.43350 | 0.00000 |
| CO₂ | | 0.00000 | 0.01261 | 0.00000 | 0.01346 | 0.07019 | 0.08858 | 0.09644 | 0.09644 | 0.00000 |
| METHANOL | | 0.00000 | 0.00000 | 0.00000 | 0.00000 | 0.00623 | 0.13301 | 0.01014 | 0.01014 | 0.83487 |
| O₂ | | 0.37107 | 0.00000 | 0.00000 | 0.00000 | 0.00000 | 0.00000 | 0.00000 | 0.00000 | 0.00000 |
| ETHANOL | | 0.00000 | 0.00000 | 0.00000 | 0.00000 | 0.00018 | 0.00678 | 0.00029 | 0.00029 | 0.03711 |
| 1-PROPANOL | | 0.00000 | 0.00000 | 0.00000 | 0.00000 | 0.00006 | 0.00347 | 0.00009 | 0.00009 | 0.02277 |

### Embodiment 3

The process of embodiment 2, wherein the syngas stream entering the reactor has a stoichiometric ratio of not greater than 1.5.

### Embodiment 4

The process of embodiment 3, wherein the syngas stream entering the reactor has a stoichiometric ratio of not greater than 1.2.

### Embodiment 5

The process of any of embodiments 1-4, wherein the syngas stream entering the reactor has a stoichiometric ratio of at least 0.1.

### Embodiment 6

The process of embodiment 5, wherein the syngas stream entering the reactor has a stoichiometric ratio of at least 0.5.

### Embodiment 7

The process of any of embodiments 1-6, wherein the catalyst comprises from 10 wt % to 70 wt % copper, on an oxide basis.

### Embodiment 8

The process of any of embodiments 1-7, wherein the catalyst comprises an oxide of zinc.

### Embodiment 9

The process of any of embodiments 1-8, wherein the catalyst comprises from 3 wt % to 40 wt % zinc oxide, based on total weight of the catalyst.

### Embodiment 10

The process of any of embodiments 1-9, wherein the catalyst comprises an oxide of aluminum.

### Embodiment 11

The process of embodiment 10, wherein the catalyst comprises from 1 wt. % to 15 wt % of an oxide of aluminum, based on total weight of the catalyst.

### Embodiment 12

The process of any of embodiments 1-11, wherein the catalyst comprises 10 wt % to 70 wt % copper, on an oxide weight basis, from 3 wt % 40 wt % zinc oxide, based on total weight of the catalyst, and from 1 wt % to 15 wt % aluminum oxide, based on total weight of the catalyst.

### Embodiment 13

The process of embodiment 12, wherein the copper and zinc are present at a Cu:Zn atomic ratio of from 0.5:1 to 20:1.

### Embodiment 14

The process of any of embodiments 1-13, wherein the catalyst comprises at least one alkali or alkaline earth metal.

### Embodiment 15

The process of any of embodiments 1-14, wherein the catalyst comprises at least one alkali metal selected from the group consisting of lithium, sodium, potassium, rubidium, cesium and francium.

### Embodiment 16

The process of any of embodiments 1-15, wherein the catalyst comprises from 0.1 wt % to 2 wt % of an alkali or alkaline earth metal, based on total weight of the catalyst.

### Embodiment 17

The process of any of embodiments 1-16, wherein the catalyst comprises lithium.

### Embodiment 18

The process of any of embodiments 1-17, wherein the catalyst comprises at least one alkaline earth metal selected from the group consisting of calcium, barium, strontium and radium.

### Embodiment 19

The process of embodiment 18, wherein the catalyst comprises calcium.

### Embodiment 20

The process of any of embodiments 1-19, wherein the alcohol is contacted with a molecular sieve catalyst to form an olefin product.

### Embodiment 21

The process of embodiment 20, wherein at least one olefin component of the olefin product is contacted with a polymer forming catalyst to form a polyolefin product.

### Embodiment 22

A catalyst composition, comprising:

10 wt % to 70 wt % copper, on an oxide weight basis,

3 wt % 40 wt % zinc oxide, based on total weight of the catalyst,

1 wt % to 15 wt % aluminum oxide, based on total weight of the catalyst; and

at least one alkali metal,

wherein the copper and zinc are present at a Cu:Zn atomic ratio of from 0.5:1 to 20:1.

### Embodiment 23

The catalyst of embodiment 22, wherein the alkali metal is selected from the group consisting of lithium and sodium.

### Embodiment 24

The catalyst of embodiment 22, wherein the alkali metal is lithium.

### Embodiment 25

The catalyst of any of embodiments 22-24, wherein the catalyst comprises 15 wt % to 68 wt % copper.

### Embodiment 26

The catalyst of embodiment 25, wherein the catalyst comprises 20 wt % to 65 wt % copper.

### Embodiment 27

The catalyst of any of embodiments 22-26, wherein the catalyst comprises 4 wt % to 35 wt % zinc oxide.

### Embodiment 28

The catalyst of embodiment 27, wherein the catalyst comprises 5 wt % to 30 wt % zinc oxide.

### Embodiment 29

The catalyst of any of embodiments 22-28, wherein the catalyst comprises 1.5 wt % to 12 wt % aluminum oxide.

### Embodiment 30

The catalyst of embodiment 20, wherein the catalyst comprises 2 wt % to 10 wt % aluminum oxide.

### Embodiment 31

The catalyst of embodiment 30, wherein the catalyst comprises 3 wt % to 8 wt % aluminum oxide.

### Embodiment 32

The catalyst of embodiment 31, wherein the catalyst comprises 4 wt % to 6 wt % aluminum oxide.

### Embodiment 33

The catalyst of any of embodiments 22-32, wherein the copper and zinc are present at a Cu:Zn atomic ratio of from 0.7:1 to 15:1.

### Embodiment 34

The catalyst of embodiment 33, wherein the copper and zinc are present at a Cu:Zn atomic ratio of from 0.8:1 to 5:1.

### Embodiment 35

The catalyst of embodiment 34, wherein the copper and zinc are present at a Cu:Zn atomic ratio of from 1.5:1 to 2.5:1.

### Embodiment 36

The catalyst of any of embodiments 22-35, wherein the alkali metal is selected from the group consisting of lithium and sodium, and the catalyst comprises 0.1 wt % to 2 wt % of the alkali metal.

What is claimed is:

1. A process for producing alcohol from syngas, comprising:
sending a syngas stream to an alcohol synthesis reactor, wherein the syngas stream entering the reactor has a stoichiometric molar ratio of less than 2; and
contacting the syngas in the alcohol synthesis reactor with a catalyst comprising copper and an oxide of at least one element selected from the group consisting of silver, zinc, boron, magnesium, aluminum, vanadium, chromium, manganese, gallium, palladium, osmium and zirconium, to form an alcohol composition containing methanol and ethanol.

2. The process of claim 1, wherein the syngas stream entering the reactor has a stoichiometric ratio of less than 1.9.

3. The process of claim 2, wherein the syngas stream entering the reactor has a stoichiometric ratio of not greater than 1.5.

4. The process of claim 3, wherein the syngas stream entering the reactor has a stoichiometric ratio of not greater than 1.2.

5. The process of claim 1, wherein the syngas stream entering the reactor has a stoichiometric ratio of at least 0.1.

6. The process of claim 5, wherein the syngas stream entering the reactor has a stoichiometric ratio of at least 0.5.

7. The process of claim 1, wherein the catalyst comprises from 10 wt % to 70 wt % copper, on an oxide basis.

8. The process of claim 1, wherein the catalyst comprises an oxide of zinc.

9. The process of claim 8, wherein the catalyst comprises from 3 wt % to 40 wt % zinc oxide, based on total weight of the catalyst.

10. The process of claim 1, wherein the catalyst comprises an oxide of aluminum.

11. The process of claim 10, wherein the catalyst comprises from 1 wt. % to 15 wt % of an oxide of aluminum, based on total weight of the catalyst.

12. The process of claim 1, wherein the catalyst comprises 10 wt % to 70 wt % copper, on an oxide weight basis, from 3 wt % 40 wt % zinc oxide, based on total weight of the catalyst, and from 1 wt % to 15 wt % aluminum oxide, based on total weight of the catalyst.

13. The process of claim 12, wherein the copper and zinc are present at a Cu:Zn atomic ratio of from 0.5:1 to 20:1.

14. The process of claim 1, wherein the catalyst comprises at least one alkali or alkaline earth metal.

15. The process of claim 1, wherein the catalyst comprises at least one alkali metal selected from the group consisting of lithium, sodium, potassium, rubidium, cesium and francium.

16. The process of claim 1, wherein the catalyst comprises from 0.1 wt % to 2 wt % of an alkali or alkaline earth metal, based on total weight of the catalyst.

17. The process of claim 1, wherein the catalyst comprises lithium.

18. The process of claim 1, wherein the catalyst comprises at least one alkaline earth metal selected from the group consisting of calcium, barium, strontium and radium.

19. The process of claim 18, wherein the catalyst comprises calcium.

20. The process of claim 1, wherein the alcohol is contacted with a molecular sieve catalyst to form an olefin product.

21. The process of claim 20, wherein at least one olefin component of the olefin product is contacted with a polymer forming catalyst to form a polyolefin product.

22. A process for producing olefin from syngas, comprising:
sending a syngas stream to an alcohol synthesis reactor, wherein the syngas stream entering the reactor has a stoichiometric molar ratio of less than 2;
contacting the syngas in the alcohol synthesis reactor with a catalyst comprising copper and an oxide of at least one element selected from the group consisting of silver, zinc, boron, magnesium, aluminum, vanadium, chromium, manganese, gallium, palladium, osmium and zirconium, to form an alcohol composition containing methanol and ethanol; and
contacting the alcohol composition with a molecular sieve catalyst to form an olefin product.

23. The process of claim 22, wherein the syngas stream entering the reactor has a stoichiometric ratio of less than 1.9.

24. The process of claim 23, wherein the syngas stream entering the reactor has a stoichiometric ratio of not greater than 1.5.

25. The process of claim 24, wherein the syngas stream entering the reactor has a stoichiometric ratio of not greater than 1.2.

26. The process of claim 22, wherein the syngas stream entering the reactor has a stoichiometric ratio of at least 0.1.

27. The process of claim 26, wherein the syngas stream entering the reactor has a stoichiometric ratio of at least 0.5.

28. The process of claim 22, wherein the catalyst comprises from 10 wt % to 70 wt % copper, on an oxide basis.

29. The process of claim 22, wherein the catalyst comprises an oxide of zinc.

30. The process of claim 29, wherein the catalyst comprises from 3 wt % to 40 wt % zinc oxide, based on total weight of the catalyst.

31. The process of claim 22, wherein the catalyst comprises an oxide of aluminum.

32. The process of claim 31, wherein the catalyst comprises from 1 wt. % to 15 wt % of an oxide of aluminum, based on total weight of the catalyst.

33. The process of claim 22, wherein the catalyst comprises 10 wt % to 70 wt % copper, on an oxide weight basis, from 3 wt % 40 wt % zinc oxide, based on total weight of the catalyst, and from 1 wt % to 15 wt % aluminum oxide, based on total weight of the catalyst.

34. The process of claim 33, wherein the copper and zinc are present at a Cu:Zn atomic ratio of from 0.5:1 to 20:1.

35. The process of claim 22, wherein the catalyst comprises at least one alkali or alkaline earth metal.

36. The process of claim 22, wherein the catalyst comprises at least one alkali metal selected from the group consisting of lithium, sodium, potassium, rubidium, cesium and francium.

37. The process of claim 22, wherein the catalyst comprises from 0.1 wt % to 2 wt % of an alkali or alkaline earth metal, based on total weight of the catalyst.

38. The process of claim 22, wherein the catalyst comprises lithium.

39. The process of claim 22, wherein the catalyst comprises at least one alkaline earth metal selected from the group consisting of calcium, barium, strontium and radium.

40. The process of claim 22, wherein the catalyst comprises calcium.

41. A catalyst composition, comprising:
10 wt % to 70 wt % copper, on an oxide weight basis,
3 wt % 40 wt % zinc oxide, based on total weight of the catalyst,
1 wt % to 15 wt % aluminum oxide, based on total weight of the catalyst; and
at least one alkali metal,
wherein the copper and zinc are present at a Cu:Zn atomic ratio of from 0.5:1 to 20:1.

42. The catalyst of claim 41, wherein the alkali metal is selected from the group consisting of lithium and sodium.

43. The catalyst of claim 41, wherein the alkali metal is lithium.

44. The catalyst of claim 41, wherein the catalyst comprises 15 wt % to 68 wt % copper.

45. The catalyst of claim, 44, wherein the catalyst comprises 20 wt % to 65 wt % copper.

46. The catalyst of claim 41, wherein the catalyst comprises 4 wt % to 35 wt % zinc oxide.

47. The catalyst of claim 46, wherein the catalyst comprises 5 wt % to 30 wt % zinc oxide.

48. The catalyst of claim 41, wherein the catalyst comprises 1.5 wt % to 12 wt % aluminum oxide.

49. The catalyst of claim 48, wherein the catalyst comprises 2 wt % to 10 wt % aluminum oxide.

50. The catalyst of claim 49, wherein the catalyst comprises 3 wt % to 8 wt % aluminum oxide.

51. The catalyst of claim 50, wherein the catalyst comprises 4 wt % to 6 wt % aluminum oxide.

52. The catalyst of claim 41, wherein the copper and zinc are present at a Cu:Zn atomic ratio of from 0.7:1 to 15:1.

53. The catalyst of claim 52, wherein the copper and zinc are present at a Cu:Zn atomic ratio of from 0.8:1 to 5:1.

54. The catalyst of claim 53, wherein the copper and zinc are present at a Cu:Zn atomic ratio of from 1.5:1 to 2.5:1

55. The catalyst of claim 41, wherein the alkali metal is selected from the group consisting of lithium and sodium, and the catalyst comprises 0.1 wt % to 2 wt % of the alkali metal.
